(19) United States
(12) Patent Application Publication
(10) Pub. No.: US 2019/0357977 A1
(43) Pub. Date: Nov. 28, 2019

(54) ULTRAFAST LASER SYSTEM AND APPLICATION THEREOF

(71) Applicants: Inno Laser Technology Corporation Limited, Shenzhen (CN); Changzhou Inno Machining Co., Ltd., Changzhou (CN)

(72) Inventors: Xiaojie Zhao, Ronkonkoma, NY (US); Sha Tao, Shenzhen (CN); Guoshuang Qin, Shenzhen (CN)

(21) Appl. No.: 16/244,079

(22) Filed: Jan. 9, 2019

Related U.S. Application Data
(63) Continuation of application No. PCT/CN2018/106857, filed on Sep. 21, 2018.

(30) Foreign Application Priority Data
May 23, 2018 (CN) 201810501341.5

(51) Int. Cl. A61B 18/20; G02B 26/10

(52) U.S. Cl. CPC A61B 18/203; A61B 2018/0019; G02B 26/105

(57) ABSTRACT

An ultrafast laser system and application thereof, relates to the technical field of laser, and the system comprises a control module, a laser, an optical path module, a movable platform, a visual locating module, and the control module is connected to the laser and controls an operation of the laser, and the control module is connected to the movable platform and controls a movement of the movable platform, and the optical path module is connected to the laser and is used for transmitting a laser light generated by the laser to the movable platform, and the control module is connected to the visual locating module and controls the visual locating module to position a laser emitting position at a position, and the laser is an ultrafast laser. The system is applied to equipment for removing mole, freckle, or tattoo, which has the advantages of fewer traumas and no pain.

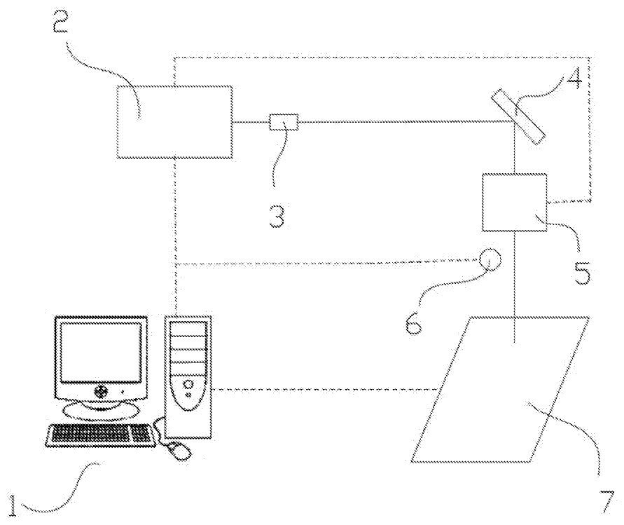

### CROSS REFERENCE TO RELATED APPLICATION

The present application is a Continuation Application of PCT Application No. PCT/CN2018/106857, filed on Sep. 21, 2018, which claims the benefit of Chinese Patent Application No. 201810501341.5 filed on May 23, 2018. All the above are hereby incorporated by reference.

### FIELD OF THE INVENTION

The present application relates to the technical field of laser, and more particularly, to an ultrafast laser system and application thereof.

### BACKGROUND OF THE INVENTION

In the prior art, lasers are gradually being applied to the biomedical field. But most applications are limited to the application of nanosecond laser or sub-nanosecond laser. Up to now, there is no ultrafast laser system which could be applied to the biomedical field in the prior art.

### SUMMARY OF THE INVENTION

The embodiments of the present application provide a novel ultrafast laser system.

An ultrafast laser system, comprising: a control module, a laser, an optical path module, a movable platform, a visual locating module, and the control module is connected to the laser and controls an operation of the laser, and the control module is connected to the movable platform and controls a movement of the movable platform, and the optical path module is connected to the laser and is used for transmitting a laser light generated by the laser to the movable platform, and the control module is connected to the visual locating module and controls the visual locating module to position a laser emitting position at a position, and the laser is an ultrafast laser.

Further, the optical path module comprises a beam expander, a reflector and a vibrating mirror in sequence according to an order of laser transmission.

Further, the laser is further connected to the vibrating mirror.

Further, the laser is a laser of which sub-pulse energy, a quantity, or a time interval is adjustable.

Further, the laser is a laser of which a wavelength is adjustable, and the wavelength of the laser is adjusted as needed.

Further, the wavelength of the laser is in a range of 500 to 1100 nm.

Further, a pulse width of the laser is less than 10 ps.

Finally, the embodiments of the present application provide an application of the ultrafast laser system, wherein the ultrafast laser system is applied to equipment for removing mole, freckle, or tattoo.

The ultrafast laser system of the embodiments of the present application utilizes the ultrafast laser. The system could be applied to equipment such as the equipment for removing mole, the equipment for removing freckle, or the equipment for removing tattoo, and compared with a conventional related equipment, the equipment comprising the system has advantages of fewer traumas and no pain.

### BRIEF DESCRIPTION OF THE DRAWINGS

The specific solution of the present application will be described in detail below with reference to the accompanying drawings.

The figure is a structural schematic diagram of an ultrafast laser system of an embodiment of the present application.

Wherein, 1-Control module, 2-Laser, 3-Beam expander, 4-Reflector, 5-Vibrating mirror, 6-Visual locating module, 7-Movable platform.

### DESCRIPTION OF THE EMBODIMENTS

In order to describe the technical content, structural features, achieved objects and effects of the present application in detail, the detailed description will be described with reference to the embodiments and the accompanying drawings.

Referring to the figure, an ultrafast laser system comprises a control module 1, a laser 2, an optical path module, a movable platform 7, and a visual locating module 6, and the control module 1 is connected to the laser 2 and controls the operation of the laser 2, and the control module 1 is connected to the movable platform 7 and controls the movement of the movable platform 7, and the movable platform 7 is mainly used for a human body to lie on, and then the movable platform 7 could be adjusted as needed, so that the laser could be aimed at a specific part. The optical path module is connected to the laser 2 and transmits a laser light generated by the laser 2 to the movable platform 7, and the control module 1 is connected to the visual locating module 6 and controls the visual locating module 6 to position the laser emitting position at a specific position, so that the laser could be accurately positioned to a specific part of the human body, and the laser 2 is an ultrafast laser. The system could be applied to equipment such as equipment for removing mole, equipment for removing freckle, or equipment for removing tattoo, etc. Compared with conventional related equipment, the system has the advantages of fewer traumas and no pain.

Preferably, the optical path module comprises a beam expander 3, a reflector 4 and a vibrating mirror 5 in sequence according to the order of laser transmission. That is, the laser 2 generates a laser light, which could be sequentially transmitted to the movable platform 7 by the beam expander 3, the reflector 4 and the vibrating mirror 5, and in combination with the visual locating system, the laser light could precisely illuminate a specific part of the human body, such as the skin part of the human body with mole, freckle or tattoo, and perform a laser removal for the part of the human body. Furthermore, the laser 2 is also connected to the vibrating mirror 5 to acquire and control the scanning of the vibrating mirror 5.

Preferably, the laser 2 is a laser of which the sub-pulse energy, quantity, and time interval are adjustable. According to the optical characteristics of the object to be removed (such as mole, freckle or tattoo), the specific parameters could be selected or adjusted to optimize the removal efficiency and quality.

Preferably, the laser is a laser of which the wavelength is adjustable, and the laser wavelength could be adjusted as needed. And the wavelength of the laser is preferably 500 to 1100 nm.

Preferably, a pulse width of the laser is less than 10 picoseconds.

In addition, the ultrafast laser system described above could be applied to equipment such as equipment for removing mole, freckle, tattoo, etc., and could be used for removing mole, freckle or tattoo removal. For example, if remove a tattoo, the application method comprises the following steps:

S1, determining a size of the tattoo area to be removed, for example, determining the size of the tattoo area on the surface of the human skin by estimation or measurement, etc.;

S2, determining an energy of the laser according to the size of the tattoo area to be removed. After obtaining the size of the tattoo area on the human skin to be removed, determine the energy of the laser needed to illuminate according to the size, for example, calculate by conversion according to the amount of laser energy required for removing tattoo per unit area, and at the same time, according to the bearing characteristics of the human skin, ensure that the energy of the laser used does not exceed the safe range, preventing the human skin from being damaged during the process of removing the tattoo;

S3, adjusting a wavelength, a pulse width, a burst parameter, a scan path, and the like of a laser beam of the ultrafast laser according to the energy of the laser determined;

S4, removing the tattoo by the ultrafast laser using the adjusted laser beam.

Meanwhile, in the above steps, it is also necessary to position the emitting laser by the visual locating system, and adjust and move the position of the platform to adjust the specific part of the human skin at which the laser is aimed.

Overall, the ultrafast laser system of the embodiment is applied to remove the mole, freckle, or tattoo of human skin by laser, which has the advantages of rapidness, fewer traumas, no pain, and the like, and not only has high overall removal efficiency, but also has good removal effect.

It should be noted that, here, the up, down, left, right, front and back only represent their relative positions, and do not represent their absolute positions. The above description is only the embodiments of the present application, and is not intended to limit the scope of the present application. The equivalent structures or equivalent flow transformations, or the directly or indirectly application in other related technology field according to the specification and the accompanying drawings should all fall into the protection scope of the present application.

What is claimed is:

1. An ultrafast laser system, comprising: a control module, a laser, an optical path module, a movable platform, a visual locating module, and the control module is connected to the laser and controls an operation of the laser, and the control module is connected to the movable platform and controls a movement of the movable platform, and the optical path module is connected to the laser and is used for transmitting a laser light generated by the laser to the movable platform, and the control module is connected to the visual locating module and controls the visual locating module to position a laser emitting position at a position, and the laser is an ultrafast laser.

2. The ultrafast laser system according to claim 1, wherein the optical path module comprises a beam expander, a reflector and a vibrating mirror in sequence according to an order of laser transmission.

3. The ultrafast laser system according to claim 2, wherein the laser is further connected to the vibrating mirror.

4. The ultrafast laser system according to claim 1, wherein the laser is a laser of which sub-pulse energy, a quantity, or a time interval is adjustable.

5. The ultrafast laser system according to claim 4, wherein the laser is a laser of which a wavelength is adjustable, and the wavelength of the laser is adjusted as needed.

6. The ultrafast laser system according to claim 5, wherein the wavelength of the laser is in a range of 500 to 1100 nm.

7. The ultrafast laser system according to claim 6, wherein a pulse width of the laser is less than 10 ps.

8. An application of the ultrafast laser system according to claim 1, wherein the ultrafast laser system is applied to equipment for removing mole, freckle, or tattoo.
